(19) United States
(12) Patent Application Publication
(10) Pub. No.: US 2005/0154069 A1
(43) Pub. Date: Jul. 14, 2005

(54) Fischer-Tropsch process in the presence of nitrogen contaminants

(75) Inventors: Juan Inga, Sapulpa, OK (US); Paul Kennedy, Tulsa, OK (US); Stephen Leviness, Tulsa, OK (US)

Correspondence Address: Valerie K. Friedrich, Ph.D.;Jenkens & Gilchrist, PC, Suite 2700, 1401 McKinney Street, Houston, TX, 77010, US

(73) Assignee: Syntroleum Corporation, Tulsa, OK (US)

(21) Appl. No.: 10/755,942

(22) Filed: Jan. 13, 2004

(51) Int. Cl. C07C 27/26

(52) U.S. Cl. 518/726

(57) ABSTRACT

A Fischer-Tropsch process in the presence of nitrogen is provided wherein the Fischer-Tropsch catalyst retains at least 50% of its original activity and about the original C₅₊ selectivity. A process for pre-conditioning a Fischer-Tropsch catalyst such that no more than 50% of the original catalyst activity is lost while the resultant catalyst retains about its original C₅₊ selectivity.

### CROSS REFERENCE TO RELATED APPLICATIONS

Not applicable.

### FEDERALLY SPONSORED RESEARCH

Not applicable.

### REFERENCE TO MICROFICHE APPENDIX

Not applicable.

### FIELD OF THE INVENTION

This invention relates to a Fischer-Tropsch process conducted in the presence of nitrogen-containing contaminants (herein referred to as “N-contaminants”), including ammonia and hydrogen cyanide. This invention relates more particularly, to a Fischer-Tropsch process in the presence of greater than 100 ppb N-contaminants wherein the poisoned Fischer-Tropsch catalyst displays improved C₅₊ selectivity. The invention further relates to a process for pre-conditioning a Fischer-Tropsch catalyst by partial, rapid poisoning with N-contaminants. The invention further relates to a process for increasing the C₅₊ yield ratio.

### BACKGROUND OF THE INVENTION

Synthesis gas (“syngas”) typically contains trace nitrogen-containing compounds, principally ammonia and hydrogen cyanide. Other reactive nitrogen compound species, such as cyanogen and nitrogen oxides, may also be present in very small amounts. Collectively, these nitrogen-containing compounds are referred to herein as N-contaminants.

N-contaminants arise from the presence of one or more nitrogen-containing species in the feed to the synthesis gas generator. For example, N₂ may be present in: (1) the feed natural gas; (2) the O₂ feed after air separation for an oxygen blown syngas generation process; and/or (3) the air feed for an air blown process. In addition to or alternatively to these sources of N₂, nitrogen-containing hydrocarbon species (especially for liquid and/or solid syngas generation feedstocks, such as residual oil or coal) may also be present in the syngas generator. The concentration of N-contaminants produced in the syngas generator may also be increased substantially through the recycle of Fischer-Tropsch tail gas into the syngas generation process. Similarly, the concentration of N-contaminants produced in the syngas generator may also be increased by recycling of tail gases from other processes into the syngas generator.

Virtually all commercially practiced and proposed syngas generation processes operate at extremely high temperatures, generally in the range of 1500°-2500° F., where the majority of the chemical reactions occur near or at chemical thermodynamic equilibrium. Under these conditions, small quantities of hydrogen cyanide (HCN) and ammonia (NH₃) are typically produced. Yet smaller amounts of other reactive nitrogen-containing compounds, such as cyanogen, may also be produced. The amounts of HCN and NH₃ in a syngas depends strongly on both the nitrogen concentration in the syngas generator feed and the process conditions, particularly pressure and temperature. Typical concentrations of these nitrogen-containing compounds in the syngas generator outlet stream which has not been further processed (referred to herein as a “raw synthesis gas”) are in the range from about 1 to about 50 vppm HCN and from about 5 to about 1000 vppm NH₃. Generally, the raw syngas contains between about 10 and about 30 times more NH₃ than HCN.

Ammonia, which is basic, is very soluble in water. Raw syngases contain both carbon dioxide and water vapor and at least about 90 wt % of the ammonia present in the raw syngas can be removed by cooling the raw synthesis gas to less than about 200° F. and condensing the produced water. CO₂ dissolved in the condensed water will facilitate dissolution of the ammonia from the synthesis gas. The amount of ammonia in the syngas may be further decreased by use of a water scrubber.

HCN, on the other hand, is much less water soluble than NH₃, and is somewhat acidic in solution. Therefore, HCN is much more difficult to remove by means of raw synthesis gas water knockouts and/or subsequent scrubbing. Removal by water scrubbing requires relatively large quantities of water, typically greater than 1:1 water:syngas mass ratios. Incremental HCN removal can be realized by recirculating the ammonia-containing wash water, produced by scrubbing the ammonia from the raw syngas which contributes to HCN disassociation and removal by water scrubbing. However, HCN removal with water scrubbing is inefficient, requiring excessive amounts of water in relation to the HCN quantity removed. A large number of known processes for HCN removal from synthesis gases, including HCN adsorption, catalytic conversion of HCN (hydrogenation and/or hydrolysis), and chemically treated water scrubbing of HCN are known. Other processes attempt to prevent the formation of HCN by upstream removal of N₂ from natural gas. Such known processes, however, result in or require increased plant capital and/or operating costs, supply and disposal of treatment chemicals, and/or potential contamination of the treated synthesis gas.

Removal of HCN and NH₃ from syngas is considered important because these nitrogen-containing compounds are poisons of Fischer-Tropsch catalysts, particularly non-shifting catalysts, and more particularly, those Fischer-Tropsch catalysts containing cobalt. Thus, current processes target maximum HCN and/or NH₃ concentrations lower than 100 ppb, lower than 50 ppb and as low as 20 ppb. The lower HCN and/or NH₃ levels are required so as to achieve a Fischer-Tropsch catalyst “half-life” (the time to lose half of the initial catalyst activity) of greater than 10 days. Thus, even when the N-contaminant level is reduced there is still some catalyst poisoning and deactivation.

Productivity is defined herein as the standard volume of carbon monoxide converted/volume catalyst/hour calculated as the product of the feed gas rate per volume of catalyst, the mole fraction of CO in the feed gas, and fractional conversion of CO. As the catalyst is poisoned, i.e. deactivated, the fractional CO conversion decreases and assuming all other reaction variables, e.g. temperature and gas hourly space velocity (“GHSV”), are held constant, the productivity decreases. This tendency is true for all reactor types.

To counteract the decreased production effect of such catalyst poisoning and deactivation, currently known processes adjust the reactor conditions in order to achieve target hydrocarbon production levels. The principal adjustments include: 1) decreasing the feed synthesis gas flow rate; 2) increasing the amount of catalyst; and/or 3) increasing the reactor temperature. The first option, however, is unpractical as it would require additional reactors to maintain an acceptable production rate. The second option, the most likely implemented, also requires increased catalyst loading. While the third option avoids these economic debits, it is accompanied by an increase in undesirable light hydrocarbon gas, i.e. C₄, production and a decrease in the average liquid hydrocarbon, i.e. C₅₊, product.

N-contaminant poisoning of Fischer-Tropsch catalysts, however, is readily reversible by hydrogen treatment at or near typical Fischer-Tropsch reaction conditions. Numerous processes to rejuvenate or regenerate slurry Fischer-Tropsch catalyst with hydrogen, which restore catalyst activity to values approaching the initial catalyst activity, are known.

Hydrogen treatment, i.e. reduction, to rejuvenate and/or oxidation and re-reduction to regenerate Fischer-Tropsch catalysts may be conducted by treating the entire reactor at once either offline or cyclically. Alternatively, continuous rejuvenation or regeneration, whereby a smaller amount of catalyst slurry is rejuvenated or regenerated in a restricted zone inside or outside the reactor may be used. Such continuous operations require special plant hardware. Both continuous and offline rejuvenation or regeneration require potentially large amounts of “practically CO free” hydrogen, which also requires specialized equipment.

It would, therefore, be desirable to maintain C₅₊ hydrocarbon production rates at about initial C₅₊ production rates in the Fischer-Tropsch synthesis without the requirement of nearly complete nitrogen (i.e. to as low as 20 ppb HCN and/or NH₃) removal while maintaining acceptable % CO conversion. Moreover, it would be desirable to minimize or eliminate the need for catalyst rejuvenation or regeneration.

### SUMMARY OF THE INVENTION

The invention provides a Fischer-Tropsch process conducted in the presence of greater than or equal to about 100 ppb N-contaminants. The inventive process further provides a CO conversion of at least between about 50% and 65% of an initial unpoisoned CO conversion rate. The inventive process further provides a C₅₊ selectively at least about equal to an initial unpoisoned C₅₊ selectivity.

The invention further provides a process to pre-condition a non-shifting Fischer-Tropsch catalyst to produce a pre-conditioned catalyst which provides at least about the same %CO conversion and C₅₊ selectivity, at elevated temperatures, as that of the same catalyst which has not been pre-conditioned operating under unpoisoned conditions and at lower temperatures.

### BRIEF DESCRIPTION OF THE DRAWINGS

None.

### DESCRIPTION OF EMBODIMENTS OF THE INVENTION

The following definitions are provided for the purposes of this disclosure and claims contained therein.

The term C₅₊ and “liquid hydrocarbons” are used synonymously and refer to hydrocarbons having five (5) or greater carbons, including for example pentane, hexane, heptane, pentanol, pentene, and which are liquid at normal atmospheric conditions.

The terms C₄₋ and “gaseous hydrocarbons” are used synonymously and refer to hydrocarbons having four (4) or fewer carbons, including for example methane, ethane, propane, butane, butanol, butene, propene, and which are gaseous at normal atmospheric conditions.

The term “non-shifting Fischer-Tropsch catalyst” refers to Fischer-Tropsch synthesis catalysts for which little or no water-gas shift occurs, i.e., less than 5% CO₂ make.

The Fischer-Tropsch reaction for converting syngas, which is composed primarily of carbon monoxide (CO) and hydrogen gas (H₂), may be characterized by the following general reaction:
2nH₂+nCO→(—CH₂—)ₙ+nH₂O (1)
Non-reactive components, such as nitrogen, may also be included or mixed with the syngas.

The syngas is delivered to a synthesis unit, which includes a Fischer-Tropsch reactor (FTR) containing a non-shifting Fischer-Tropsch catalyst. Numerous non-shifting Fischer-Tropsch catalysts may be used in carrying out the reaction. These include cobalt, ruthenium as well as other Group VIIIB transition metals or combinations of such metals, to prepare both saturated and unsaturated hydrocarbons. The non-shifting Fischer-Tropsch catalyst may include a support, such as a metal-oxide support, including silica, alumina, silica-alumina or titanium oxides. For example, a Co catalyst on alumina with a surface area of about 100 to about 200 m²/g may be used in the form of spheres of about 20 to about 150 μm in diameter. The Co concentration on the support may be from about 15% to about 30%. Certain catalyst promoters and stabilizers may be used. The stabilizers include Group IIA or Group IIIB metals, while the promoters may include elements from Group VIII or Group VIIB. The non-shifting Fischer-Tropsch catalyst and reaction conditions may be selected to be optimal for desired reaction products, such as for hydrocarbons of certain chain lengths or number of carbon atoms. Any of the following reactor configurations may be employed for Fischer-Tropsch synthesis: fixed bed, slurry bed reactor, ebullating bed, fluidized bed, or continuously stirred tank reactor (CSTR). The FTR may be operated at a pressure of about 100 to about 600 psia and a temperature of about 375° F. to about 500° F. The reactor gas hourly space velocity (“GHSV”) may be from about 1000 to about 15000 hr⁻¹. Syngas useful in producing a Fischer-Tropsch product useful in the invention may contain gaseous hydrocarbons, hydrogen, carbon monoxide and nitrogen with H₂/CO ratios from about 1.8 to about 2.4. The hydrocarbon products derived from the Fischer-Tropsch reaction may range from methane (CH₄) to high molecular weight paraffinic waxes containing more than 100 carbon atoms.

In one embodiment of the invention, a Fischer-Tropsch synthesis is conducted so as maximize the production of hydrocarbon liquids, C₅₊ hydrocarbons, while minimizing or eliminating N-contaminant removal from the syngas prior to feed into the Fischer-Tropsch reactor. The Fischer-Tropsch synthesis process includes feeding a raw synthesis gas into an FTR containing an active non-shifting Fischer-Tropsch catalyst under initial reaction conditions, including an initial syngas feed rate (Fi), an initial temperature (Ti), and an initial pressure (Pi). The initial reaction conditions are selected to achieve an initial target % CO conversion (Ci) with an initial liquid hydrocarbon selectivity (Li). Because there is no or minimal removal of N-contaminants from the raw syngas, the FTR feed contains greater than 100 ppb N-contaminants and a rapid deactivation of the non-shifting Fischer-Tropsch catalyst occurs, with the % CO conversion dropping to between about 55% and 65% of its initial value, Ci. Thereafter, the temperature is raised to a new temperature (Tn) such that the % CO conversion is returned to about its original value, Ci. After raising the temperature to Tn, the liquid hydrocarbon selectivity is at least about its initial value, Li.

In another embodiment of the invention, a non-shifting Fischer-Tropsch catalyst is pre-conditioned by contacting with syngas having at least about 100 ppb N-contaminants. The pre-conditioning step may be conducted within an FTR in which the pre-conditioned catalyst will be used or may be occur in a separate vessel or reactor. The pre-conditioned catalyst may then be used in an FTR at temperatures elevated over that used with a non-pre-conditioned catalyst such that a % CO conversion about the same as that of a non-pre-conditioned catalyst is obtained. The pre-conditional catalyst will provide at least about C₅₊ selectivity of a non-pre-conditional catalyst at about the same % Co conversion.

In another embodiment of the invention, the operating pressure of the FTR is increased over that generally used. Specifically, operating pressures of between about 500 psig and about 600 psig in conjunction with the increased temperatures necessary because of catalyst poisoning permit % CO conversions and C₅₊ selectivity at least about equal to that using nonpoisoned catalysts.

In yet another embodiment of the invention, either one or both of the syngas generation product water knockout and NH₃/HCN water scrubber temperatures may be raised up to about 220° C. A raise in such temperature(s) results in an increase in the water concentration in the FTR and consequently, an increase in NH₃ and HCN concentrations. This embodiment of the invention is most appropriate where high pressure syngas generation, i.e. no compressor between the syngas generator outlet and the. FTR, is used. Expressed differently, the water to carbon monoxide ratio in the feed syngas is increased in some embodiments. In air-blown systems, the water concentration may be raised from about less than 1% to about 3%-4% with a drop in carbon monoxide concentration of from about 16% to about 15%. Other Fischer-Tropsch systems, such as O₂-blown systems, would have different starting carbon monoxide concentrations but, in such embodiments, would be subjected to similar decreases in carbon monoxide and increases in water concentrations.

In the case of a lower pressure synthesis gas generation process that requires synthesis gas compression between the synthesis gas generation and the Fischer-Tropsch synthesis reactors, the maximum water vapor concentration in the synthesis gas will typically be set by the maximum allowable compressor suction temperature, which is normally no higher than 150° F., and can be substantially lower. In addition, conditions at the compressor discharge may further reduce the water vapor concentration, especially at high compression ratios. In such embodiments, the Fischer-Tropsch reactor feed synthesis gas water vapor concentration is increased by either direct steam injection or direct gas-water contacting downstream of the syngas compressor.

In yet another embodiment of the invention, Fischer-Tropsch products incorporating the nitrogen in the N-contaminants in the feed syngas are separated from the remaining Fischer-Tropsch products. At typical Fischer-Tropsch operating conditions, some significant fraction, between about 5% and about 50% of the N-contaminants and/or the nitrogen of the N-contaminants are incorporated into the Fischer-Tropsch products, such as aliphatic amines of Fischer-Tropsch waxes. A number of methods for isolating such amines are known, and include for example, adsorption on mildly acidic solids. Alternatively, the amines may be removed by hydrotreatment upstream of hydproprocessing.

Nitrogen that is not incorporated into the hydrocarbon products typically leaves the Fischer-Tropsch reactor as NH₃ in the reactor offgas. This NH₃ will normally be nearly quantitatively removed by the very acidic Fischer-Tropsch product water in the reactor cold separator, but it may be necessary to increase the effectiveness of gas-liquid contacting in order to achieve almost complete (approaching 100%) removal. In this manner it is possible to cost effectively achieve virtually zero (<10 ppb) total N concentration in the feed to subsequent, i.e., second or third stage, Fischer-Tropsch reactors under normal operating conditions. In this embodiment, care should preferably be used during first stage reactor startups, shutdowns, and/or upsets so as to avoid inadvertent nitrogen contamination of the downstream Fischer-Tropsch reactors. Sending the first stage tail gas to the second stage should preferably be avoided when the first stage reactor is operating at low temperatures, below about 400° F., where conversion of HCN to NH₃ may not be complete. Under such conditions the tail gas is preferably flared, combusted, or recycled to syngas generation.

The loss of catalyst activity discussed herein refers to the loss associated with nitrogen poisoning. Catalyst activity is also decreased due to factors independent of nitrogen concentration in the feed gas, including, but not limited to, refractory carbon formation (i.e. coking), metal sintering (growth of metal particle size and concomitant loss of metal surface area), loss of support pore volume and/or surface area, and poisoning by other species such as sulfur and chlorine. Because of the non-nitrogen dependent loss, there may nevertheless be a need for catalyst rejuvenation or regeneration even when the process of the present invention is utilized. Moreover, there may be sufficient catalyst activity loss such that the original catalyst activity cannot be recovered while retaining C₅₊ selectivity at about its initial, non-poisoned value. Note that total productivity may also be reduced by attrition related loss of catalyst which is not recoverable by catalyst regeneration or rejuvenation.

The following examples are intended to illustrate aspects of embodiments of the invention but are not intended to limit the scope of the claims herein. In all of the examples discussed below, a promoted Cobalt catalyst on alumina was used.

### EXAMPLE 1

### Deactivation Rates in the Presence of N-contaminants

Fischer-Tropsch syntheses were conducted under the conditions shown in Table 1. The runs designated A and B were conducted in a 0.5 liter Autoclave Engineers autoclave using a continuous feed process. The runs designated C and D were conducted in the same type of autoclave also using a continuous feed process. For each of runs A, B, C & D, the autoclave contained Fischer-Tropsch wax and catalyst and synthesis gas was continuously fed to the autoclave. The runs designated E and F were conducted in a pilot plant scale operation using a continuous feed process. All of the runs utilized a pure syngas of CO, H₂, and N₂ with the N-species doping as shown in Table 1. The loss of catalyst activity due to the presence of the N-species was determined algorithmically based upon a laboratory derived kinetic model and using observed CO conversion. In runs A-F, the N-contaminant-induced deactivation results in a loss of less than 40% of the catalyst activity. Following this rapid poisoning, the deactivation rate drops precipitously despite the continued presence of the same amount of N species in the feed, and approaches the rate of deactivation observed in the substantial absence of any N-contaminants.

TABLE 1
Summary of Slurry Catalyst Poisoning Results

| | Initial Conditions | | | | | | | Final | Deactivation Rates | |
|---|---|---|---|---|---|---|---|---|---|---|
| | Prod | Temp | P | GHSV | CO | N | N | % Act | % Conv | |
| Run | (v/v/hr) | (° F.) | (Psig) | (hr⁻¹) | Conv | species | (ppm) | Loss | per hr | %/Act/hr |
| A | 444 | 410 | 305 | 6000 | 45% | NH₃ | 2.5 | 28.4 | 4.73 | 5.54 |
| B | 615 | 410 | 304 | 12000 | 31% | NH₃ | 2.5 | 37.8 | 9.32 | 10.2 |
| C | 505 | 410 | 305 | 6000 | 50% | NH₃ | 1 | 36.8 | 0.75 | 0.94 |
| D | 585 | 420 | 405 | 7000 | 50% | NH₃ | 1 | 32.1 | 0.68 | 0.95 |
| E | 590 | 429 | 400 | 6600 | 55% | HCN | 0.3 | 16.3 | Not | 0.68 |
| | | | | | | | | | Isothermal | |
| F | 460 | 424 | 416 | 4900 | 60% | HCN | 3 | 29.5 | Not | 13.9 |
| | | | | | | | | | Isothermal | |

### EXAMPLE 2

### C₅₊ Selectivity in Poisoned Conditions

A synthesis gas was fed into the pilot plant slurry bubble column reactor (“SBCR”). The SBCR is nominally 6 inches in diameter and about 30-40 feet in length. The H₂:CO molar ratio of the feed syngas was about 2.15 to 2.30. The SBCR was permitted to operate for about 20 to 30 hours under unpoisoned conditions, i.e., <about 20 ppb N-species. Thereafter, the HCN removal system was bypassed allowing about 300 ppb HCN to enter with the FTR feed. Rapid deactivation occurred thereafter. The temperature was then raised until the % CO conversion returned to about the initial % CO conversion as observed before the HCN addition. The results are shown below in table 2.

TABLE 2
Pilot Plant Results

| | Temp | Press | GHSV | | | | | |
|---|---|---|---|---|---|---|---|---|
| | (° F.) | (psig) | (hr⁻¹) | Conv (%) | % CH₄ | % C₂-C₄ | % CO₂ | % C ₅₊ |
| Before 300 ppb HCN addition | 439.0 | 400.6 | 6593 | 57.0 | 9.65 | 9.00 | 0.79 | 80.57 |
| After Addition | 446.9 | 400.4 | 6604 | 56.9 | 9.04 | 8.77 | 1.05 | 81.14 |

The rapid deactivation of the non-shifting Fischer-Tropsch catalyst by N-contaminants results in a suppression of C₄₋ gaseous hydrocarbon selectivity. That is, after the reactor temperature was increased, the C₄₋ gaseous hydrocarbon selectivities are less than at the lower temperature prior to the poisoning event, i.e. addition of 300 ppb HCN. Thus, at constant reactor catalyst loading and conditions (other than temperature), production of C₅₊ hydrocarbons was increased in the presence of about 300 ppb (0.30 ppm) HCN.

### Comparative Example 3

### Decrease in C₅₊ Selectivity under Unpoisoned Conditions

TABLE 3

| T (° F.) | GHSV (hr⁻¹) | Conv | CH₄₋ Sel | C₅₊ Sel |
|---|---|---|---|---|
| 410 | 5250 | 50.30% | 6.30% | 85.00% |
| 420 | 7250 | 50.10% | 7.90% | 84.40% |
| 430 | 8000 | 49.50% | 8.10% | 83.20% |

A Fischer-Tropsch synthesis was conducted in an autoclave as described in Example 1. The feed to the autoclave did not contain N-contaminants and the run was made at constant pressure. The results are shown above in Table 3. Note that under unpoisoned, constant pressure conditions, C₅₊ selectivity decreases rapidly with temperature.

### EXAMPLE 4

### Effect of Pressure on Light Gas and C₅₊ Selectivity

TABLE 4

| | T Avg | P Inlet | GHSV | | | | | | |
|---|---|---|---|---|---|---|---|---|---|
| Unit | (° F.) | (psig) | (hr⁻¹) | Conv | CH₄ | C₂-C₄ | C₅₊ | CO₂ | H₂/CO |
| Pilot | 419 | 290 | 3807 | 60.27 | 8.72 | 8.74 | 81.33 | 1.21 | 2.14 |
| Plant | 423 | 413 | 4749 | 61.22 | 8.65 | 8.85 | 81.46 | 1.04 | 2.18 |
| Pilot | 441 | 269 | 6585 | 59.71 | 10.29 | 9.87 | 78.63 | 1.20 | 2.13 |
| Plant | 446 | 340 | 8290 | 60.18 | 10.12 | 9.80 | 79.00 | 1.08 | 2.12 |
| | 449 | 399 | 9707 | 59.88 | 10.00 | 9.61 | 79.41 | 0.98 | 2.13 |

Two runs were made in the pilot plant under unpoisoned conditions, i.e. <about 20 ppb N-species, with a syngas feed having an H₂:CO ratio of between about 2.15 and about 2.30. Note that in each of the runs, as described in Table 4 above, the higher pressure data show higher C₅₊ selectivity despite the fact in each case the temperature is higher. That is, the effects of higher pressure more than cancel out the effects of higher temperature on C₅₊ selectivity.

### EXAMPLE 5

### Pre-conditioning with HCN to Improve C₅₊ Selectivity

TABLE 5

| | T Avg | P Inlet | GHSV | | | | | | |
|---|---|---|---|---|---|---|---|---|---|
| Sample | (° F.) | (psig) | (hr⁻¹) | Conv | CH₄ | C₂-C₄ | C₅₊ | CO₂ | H₂/CO |
| 1. | 424 | 416 | 4896 | 60.22 | 8.93 | 9.13 | 80.82 | 1.12 | 2.19 |
| 2. | 424 | 417 | 4911 | 48.22 | 7.97 | 8.31 | 82.69 | 1.03 | 2.19 |
| 3. | 428 | 417 | 4939 | 60.63 | 8.85 | 9.00 | 80.83 | 1.31 | 2.19 |

A pilot plant run was made under conditions described in Table 5. The syngas feed had an H₂:CO ratio of between about 2.15 and about 2.30. The first sample from the Fischer-Tropsch reactor, which is a slurry bubble column reactor, was collected during operation with about 10 ppb HCN in the feed syngas, i.e. unpoisoned conditions. Thereafter, the HCN removal system was bypassed and approximately 3.0 ppm (3,000 ppb) HCN entered the Fischer-Tropsch reactor for approximately 6 hours. The second sample of the FT process was then collected. Conversion was observed to drop rapidly and then flatten out during this 6 hour period. The HCN removal system was then put back in service, and the second sample of effluent from the Fischer-Tropsch reactor was obtained. Note that sample 2 was taken with operations at the same temperature as sample 1, i.e. before the poisoning event. Note the significantly higher C₅₊ selectivity (82.7 vs. 80.8) following the poisoning event. The temperature was then increased until the % CO conversion returned to about its initial value in unpoisoned conditions, about 60%, at about the initial feed rate. The temperature increase required to recapture the initial conversion was about 4° F. At this increased temperature, the third sample was collected. Note that the C₅₊ selectivity of the third sample is nearly identical to that of the first sample.

The invention provides many benefits over current processes. Some of such benefits are discussed below.

Slurry Fischer-Tropsch reactors are typically operated at heat transfer limited conditions, and usually at temperatures significantly lower than 450° F., with temperatures between about 410° and about 430° F. being more common. Operations at higher temperatures provides one or more benefits, including: 1) generation of higher pressure steam from the reactor cooling system, 2) reduction in the number of cooling tubes in the reactor, and hence reduced costs; and 3) greater absolute productivity out of a given reactor system.

Higher N-contaminant lead to higher N-incorporation in the Fischer-Tropsch product stream, as discussed above. An advantage of the presence of the Fischer-Tropsch amine products in the wax is the mitigation of the need for NH₃, or other base, addition to moderate the downstream process catalyst acidity.

1. A Fischer-Tropsch synthesis process comprising:
conducting a Fischer-Tropsch synthesis on a synthesis gas comprising greater than or equal to about 100 ppb N-contaminants in the presence of a non-shifting Fischer-Tropsch catalyst wherein the CO conversion is at least between about 50% and 65% of initial unpoisoned CO conversion activity and the C₅₊ selectivity is at least about equal to the initial unpoisoned C₅₊ selectivity.

2. The process of claim 1 wherein the N-contaminants are present in a range of from about 100 ppb to about 3 ppm.

3. The process of claim 1 wherein the N-contaminants is selected from the groups of ammonia, hydrogen cyanide, cyanogen, and mixtures of one or more of such compounds.

4. The process of claim 1 wherein the non-shifting Fischer-Tropsch catalyst comprises cobalt.

5. The process of claim 1 wherein the process occurs in a slurry reactor.

6. The process of claim 1 wherein the non-shifting catalyst is rejuvenated less frequently than in the absence of N-contaminants.

7. The process of claim 4 wherein the catalyst is a promoted cobalt catalyst on alumina.

8. A liquid hydrocarbon production process comprising:
introducing a syngas comprising less than 100 ppb N-contaminants into an FTR having a non-shifting Fischer-Tropsch catalyst at an initial temperature, Ti, wherein an initial CO conversion Ci and an initial C₅₊ selectivity, Li, are achieved;
introducing a syngas having greater than 100 ppb into the FTR at Ti until the CO conversion drops to between about 50% and 65% of Ci;
raising the temperature of the FTR to achieve a CO conversion of about equal to or greater than Ci and wherein the C₅₊ selectivity is about equal to or greater than Li.

9. The process of claim 8 wherein the N-contaminants are selected from the group of ammonia, hydrogen cyanide, cyanogen, and mixtures of one or more such compounds.

10. The process of claim 8 wherein the catalyst comprises cobalt.

11. The process of claim 8 wherein the non-shifting Fischer-Tropsch catalyst is a promoted cobalt catalyst on alumina.

12. The process of claim 8 wherein the process occurs in a slurry reactor.

13. A process to pre-condition a non-shifting Fischer-Tropsch catalyst comprising the step of exposing the catalyst to greater than 100 ppb N-contaminants.

14. The process of claim 1 further comprising the steps of:
recovering the total product of the Fischer-Tropsch synthesis; and
separating N-containing Fischer-Tropsch products from the total product.

15. The process of claim 8 further comprising the steps of:
recovering the total product of the Fischer-Tropsch synthesis; and
separating N-containing Fischer-Tropsch products from the total product.
